(19) United States
(12) Patent Application Publication
(10) Pub. No.: US 2024/0122946 A9
(43) Pub. Date: Apr. 18, 2024

(54) METHODS AND COMPOSITIONS FOR TREATING 25-HYDROXYVITAMIN D DEFICIENCY

(71) Applicant: Board of Supervisors of Louisiana State University and Agricultural and Mechanical College, Baton Rouge, LA (US)

(72) Inventor: Sushil JAIN, Shreveport, LA (US)

(21) Appl. No.: 16/733,113

(22) Filed: Jan. 2, 2020

Related U.S. Application Data
(60) Provisional application No. 62/787,448, filed on Jan. 2, 2019.

(51) Int. Cl. A61K 31/593; A61K 31/198; A61K 38/06; A61P 3/02

(52) U.S. Cl. CPC A61K 31/593; A61K 31/198; A61K 38/063; A61P 3/02

(57) ABSTRACT

A method of treating a cholecalciferol (VD) deficiency associated condition in a patient comprising administering a pharmacologically effective does of a pharmaceutical composition containing VD and one of glutathione (GSH) and a GSH precursor. A pharmaceutical composition for treating a cholecalciferol (VD) deficiency associated condition comprising a pharmacologically effective does of VD and one of glutathione (GSH) and a GSH precursor.

### CROSS REFERENCE TO RELATED APPLICATIONS/PRIORITY

The present invention claims priority to U.S. Provisional Patent Application No. 62/787,448 filed Jan. 2, 2019, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under Grant No. R01AT007442 awarded by the National Institutes of Health. The government has certain rights in the invention.

### BACKGROUND

Changes in modern lifestyles that limit physical and outdoor activity and increased consumption of high-energy diets have led to a high incidence of obesity and diabetes and widespread inadequacy/deficiency of 25-hydroxyvitamin D [25(OH)VD] in populations worldwide. 25(OH)VD deficiency/inadequacy is a major public health issue affecting more than 1 billion people worldwide. Epidemiological studies provide conclusive evidence that lower circulating levels of 25(OH)VD are associated with the poor outcomes frequently associated with several chronic metabolic diseases. This has led to widespread use of vitamin D (VD) supplements by the public attempting to achieve better health. However, randomized controlled clinical trials have shown that high supraphysiological doses of VD are needed to achieve the required levels of VD in the circulation and that not all subjects respond to vitamin D (VD) supplementation.

### SUMMARY

Wherefore, it is an object of the present invention to overcome the above-mentioned shortcomings and drawbacks associated with the current technology.

The present invention relates to pharmaceutical compositions of a therapeutic (e.g., VD and GHS/GHS precursors), which includes pharmaceutically acceptable salts, solvates, esters, amides, clathrates, stereoisomers, enantiomers, prodrugs or analogs thereof, and use of these compositions for the treatment of a VD deficiency associated condition, including VD deficiency, insulin resistance,

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is a VD deficiency associated condition.

In certain embodiments, the VD deficiency associated condition is mild to moderate VD deficiency associated condition.

In further embodiments, the VD deficiency associated condition is moderate to severe VD deficiency associated condition.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of the VD deficiency associated condition.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coaterie), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., VD and GHS/GHS precursors, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., a VD deficiency associated condition). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

The presently claimed invention relates to products and methods of treating a cholecalciferol (VD) deficiency associated condition in a patient. The method of treating also includes administering a pharmacologically effective does of a pharmaceutical composition containing VD and one of glutathione (GSH) and a GSH precursor.

Implementations may include one or more of the following features. The method where the VD deficiency associated condition is one of insulin resistance (IR), inflammation, decreased 1alpha,25-dihydroxyvitamin d3 (1,25(oh)2VD) blood level, elevated blood TNF-α level, elevated blood glucose level, elevated blood hba1c level, and one or more chronic metabolic diseases. The method where the chronic metabolic disease includes one or more of obesity, diabetes, cardiovascular disease, and liver disease. The method where the pharmaceutical composition contains a GSH precursor. The method where the GSH precursor is one of n-acetylcysteine, l-cysteine (LC), cystathionine, homocysteine, s-adenosylmethionine, and l-methionine. The method where the pharmaceutical composition is administered via one of topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, nebulized, by suppositories, and oral administration. The method where the dosage of the one of GSH and GSH precursor is between 1.0 mg/kg and 20 mg/kg body weight of patient. The method where the dosage of the one of GSH and GSH precursor is between 2.5 mg/kg and 10 mg/kg body weight of patient. The method where the dosage of the one of GSH and GSH precursor is between 4.0 mg/kg and 6.0 mg/kg body weight of patient. The method where the dosage of vc is between 0.40 μg/kg and 7.00 μg/kg body weight of patient. The method where the dosage of vc is between 0.80 μg/kg and 3.50 μg/kg body weight of patient. The method where the dosage of vc is between 1.60 μg/kg and 1.74 μg/kg body weight of patient. The method where the dosage of the one of GSH and GSH precursor is one of between 200 mg and 2000 mg, between 350 and 1000 mg, between 450 mg and 550 mg, and 500 mg.

A further general aspect includes a pharmaceutical composition for treating a cholecalciferol (VD) deficiency associated condition. The pharmaceutical composition also includes a pharmacologically effective does of VD and one of glutathione (GSH) and a GSH precursor.

Implementations may include one or more of the following features. The pharmaceutical composition where the pharmaceutical composition contains a GSH precursor. The pharmaceutical composition where the GSH precursor is one of n-acetylcysteine, l-cysteine (LC), cystathionine, homocysteine, s-adenosylmethionine, and l-methionine. The pharmaceutical composition where the pharmaceutical composition formulated for administration via one of topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, nebulized, by suppositories, and oral administration. The pharmaceutical composition where dosage of the one of GSH and GSH precursor is between 200 mg and 2000 mg. The pharmaceutical composition where dosage of the one of GSH and GSH precursor is between 350 and 1000 mg. The pharmaceutical composition where dosage of the one of GSH and GSH precursor is between 450 mg and 550 mg. Implementations of the described techniques may include hardware, a method or process, or computer software on a computer-accessible medium.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 shows blood levels of 25(OH)VD and GSH and its positive association in obese adolescent. Blood levels of GSH (a), carbonyl-protein (b), 25(OH)VD (c), VDBP (d), TNF-α (e), and HOMA-IR (f) in lean, overweight, and obese adolescents. This illustrates a significant reduction in GSH and 25(OH)VD and increase in TNF-α, carbonyl-protein, and IR levels in obese subjects, and that 25(OH)VD levels are positively correlated with the GSH status in adolescents (g). BMI was used as an additional independent variable to calculate r and p-values for the correlation between 25(OH)VD and GSH. Mean±SE; data analyzed using one-way ANOVA. 25(OH)VD, 25-hydroxyvitamin D; ANOVA, analysis of variance; BMI, body mass index; GSH, glutathione; HOMA-IR, homeostatic model assessment insulin resistance; IR, insulin resistance; TNF-α, tumor necrosis factor alpha; VDBP, vitamin D binding protein.

FIG. 2 illustrates a significant decrease in GSH and 25(OH)VD, and significantly increased carbonyl protein, TNF-α, and IR levels in the blood of HFD-fed mice compared with those of mice fed with control diet. (a) GSH, (b) Carbonyl protein, (c) 25(OH)VD, (d) VDBP, (e) TNF-α, (f) IR in the blood of HFD-fed mice compared to control group. Mean±SE (n=7); data analyzed using unpaired Student's t-test. HFD, high-fat diet.

FIG. 3 shows the effect of HFD or control diet on mRNA and protein expression of GSH regulating genes (a-c), VD regulating genes (d-f), and oxidative stress biomarker levels (g) in liver of mice. HFD caused significant downregulation of VD regulatory, reduced GSH, and increases in oxidative stress. Mean±SE (n=7); data analyzed using unpaired Student's t-test. GCLC, glutamate-cysteine ligase catalytic subunit; GCLM, glutamate-custeine ligase regulatory subunit; VD, vitamin D; VDBP, vitamin D binding protein; VDR, vitamin D receptor.

FIG. 4 shows the effect of HFD or control diet on mRNA and protein expression of GSH regulating genes (a-c), VD regulating genes (d-f), glucose metabolism genes (g-i), and oxidative stress biomarker levels (j) in skeletal muscle of mice. HFD caused significant downregulation of VD regulatory and GSH biosynthesis genes, reduced GSH, and increases in carbonyl protein and MDA levels in the skeletal muscle; and a downregulation of PGC-1α/GLUT-4 and upregulation of TNF-α in comparison with mice fed a control diet. Mean±SE (n=7); data analyzed using unpaired Student's t-test. GLUT-4, glucose transporter type 4; MDA, malondialdehyde; PGC-1α, peroxisome proliferator-activated receptor gamma coactivator 1-alpha; RXRα, retinoic X receptor.

FIG. 5 shows the experimental design for supplementation of placebo (saline, n=7), OO—control, LC (n=6), VD (n=6), and combined VD+LC (n=6). Mice were purchased at 5 weeks of age and then kept in institutional animal house for acclimatization for 1 week. Mice were then randomized into different groups and were maintained on VD-deficient HFD (to mimic VD deficiency) for 8 weeks. Then mice were also gavaged with saline, LC, OO, VD, or VD+LC for 8 weeks. VD was dissolved in OO and one group of mice also gavaged with similar amounts of OO (vehicle alone). VD versus VD+LC groups have similar amounts of VD and OO-vehicle. BW, body weight; LC, I-cysteine; OO, olive oil.

FIG. 6 shows the effect of supplementation with VD+LC (green bar) versus VD alone (red bar) on blood levels of GSH (a), carbonyl protein (b), 25 (OH)VD (c), VDBP (d), TNF-α (e), HOMA-IR (f), fasting glucose (g), and HbA_{1c} (h) in mice maintained on a VD-deficient HFD for 16 weeks. Mice were gavaged with saline, OO, LC, VD, or VD+LC during last 8 weeks. VD was dissolved in OO and one group was also gavaged with OO (vehicle) alone. This shows a significantly greater increase in GSH and 25(OH)VD, and lower TNF-α, IR, glucose, and HbA_{1c} levels in combined VD+LC compared with those supplemented with VD alone. Mean±SE (n=6); data analyzed using ANOVA Holm-Sidak method with vehicle (OO) group as a control (blue bar).

FIG. 7 shows the effect of supplementation with VD+LC (green bar) versus VD alone (red bar) on mRNA and protein expression of GSH biosynthesis genes (a-c), VD regulating genes (d-f), and oxidative stress biomarkers (g) in livers of mice maintained on a VD-deficient HFD for 16 weeks. Mice were gavaged with saline, OO, LC, VD, or VD+LC during last 8 weeks. Compared with VD alone, combined VD+LC showed a significantly greater upregulation of VD regulatory and GSH biosynthesis genes, increased GSH, and lower oxidative stress. Mean±SE (n=6) and analyzed using ANOVA Holm-Sidak method with vehicle (OO) group as a control (blue bar).

FIG. 8 shows the effect of oral supplementation with VD+LC (green bar) versus VD alone (red bar) on mRNA and protein expression of GSH biosynthesis genes (a-c), VD regulating genes (d-f), glucose metabolism genes (g-i), and oxidative stress biomarkers (j) in skeletal muscle of mice maintained on VD-deficient HFD for 16 weeks. Compared with VD alone, combined VD+LC showed a greater upregulation of VD regulatory and GSH biosynthesis genes, increased GSH, reduced oxidative stress, and a greater upregulation of PGC-1α/GLUT-4. Mean±SE (n=6); data analyzed using ANOVA Holm-Sidak method with vehicle (OO) group as a control (blue bar).

FIG. 9 shows the effect of GSH-deficiency, L-cysteine, and cholecalciferol supplementation on mouse hepatocytes. Effect of GCLC-KD on GCLC (a), and mRNA levels of VD regulatory genes (b-f). GSH deficiency dose dependently downregulated Vdbp, Cyp27a1, Cyp27b1, and Vdr, and upregulated Cyp24a1. This figure also shows that GSH deficiency decreased GSH (g) and increased MDA (h) and carbonyl-protein (i); and LC (6 h) increased GSH (j) and lowered MDA (k) and carbonyl-protein (l) in GCLC-normal and GCLC-KD hepatocytes. Effect of combined LC (300 μM, 2 h preincubation) and VD (10 nM, 22 h) on GSH (r) and mRNA levels of GCLC (m), Cyp24a1 (p), VDR (q), CYP27A1 (o), and VDBP (n) shows that LC increases GSH and upregulation of CYP27A1/CYP27B1/VDBP by VD. Mean±SE (n=3); data analyzed using one-way ANOVA. CYP, cytochrome P450 enzymes; GCLC-KD, glutamate-cysteine ligase catalytic subunit knockdown; VDR, vitamin D receptor.

FIG. 10 shows the effect of GSH-deficiency, TNF-α treatment, L-cysteine, and active vitamin D supplementation on mouse myotubes. Effect of GCLC-KD (a-f) and LC (300 μM, 6 h) (k, m-p) on mRNA expression of GCLC, TNF-α, VDR, PGC-1α, GLUT-4, and GSH in myotubes. GSH deficiency caused a significant downregulation of VDR, PGC-1α, and GLUT-4 and an increase in TNF-α in GSH-deficient cells. LC helped increase GSH levels and reversed the downregulation of VDR1α/PGC-1α/GLUT-4 and upregulation of TNF-α (k-p). TNF-α per se inhibited the VDR/PGC-1α/GLUT-4 gene expression (g-j). Results (q-s) show that the effect of AVD on upregulation of NRF2 and GLUT-4 was absent in PGC-1α KD myotubes, which indicates that PGC-1α mediates the upregulation of NRF2 and GLUT-4 by 1,25(OH)₂VD. Mean±SE (n=4) and analyzed using one-way ANOVA. AVD, active vitamin D. Cotreatment with LC and 1,25(OH)₂VD (active vitamin D) resulted in significantly greater upregulation of PGC-1α/NRF2/GLUT-4 (FIG. 10q-s) gene expression in comparison with results from treatment with 1,25(OH)₂VD alone; however, this was not seen in PGC-1α-KD cells. Thus, a reduction in levels of TNF-α by LC will result in lowered inflammation and improved glucose metabolism and skeletal muscle function. 1,25(OH)₂VD was used to understand its efficacy on glucose metabolism genes in muscle cells, whereas cholecalciferol was used with hepatocyte studies to understand 25(OH)VD biosynthesis from cholecalciferol in liver.

FIG. 11 schematically illustrates a proposed mechanism for role of GSH deficiency in 25(OH)VD deficiency and potential of combined VD and LC supplementation on stimulation of VD regulatory genes and protection from 25(OH)VD deficiency and inflammation.

FIG. 12 shows the correlation coefficients (r) among the blood levels of 25(OH)VD, BMI, carbonyl-protein, TNF-α and HOMA-insulin resistance (IR) levels in adolescents. This illustrates that 25(OH)VD levels have negative association with BMI and IR, and TNF-α has a positive association with BMI, carbonyl protein and insulin resistance levels in adolescents.

FIG. 13 shows body weight, and other blood biomarkers in mice maintained on normal diet (ND) or high fat diet (HFD) for 16 wks. Data expressed as mean±SE (n=7) and analyzed using unpaired Student's ‘t’ test. Differences in *vs** are significant (p≤0.05).

FIG. 14 shows the effect of supplementation of cholecalciferol (VD) along with L-cysteine (LC) on body weight, and of the blood biomarkers in mice maintained on VD-deficient-HFD (to mimic VD-deficiency) for 8 wks; and then in addition gavaged daily for another 8 wks. Data expressed as mean±SEM (n=6) and analyzed using ANOVA Holm-Sidak method with vehicle-(OO) group as a control. Differences in values (±SE) *vs**, are significant (p<0.05).

FIG. 15 shows details of primer used in the inventors' experiments.

FIG. 16 shows the effect of Vdr-knock down on mRNA expression levels of Vdbp, Cyp27a1 and Vdr in combined LC and cholecalciferol treated hepatocytes. Effect of activation of LC on Vdbp and Cyp27a1 was significantly inhibited in Vdr-KD hepatocytes. ±SEM (n=3); data analyzed using one Way ANOVA.

FIG. 17 shows control (n=7) and high fat diet-fed mice (n=7) experimental design. Mice were purchased at 5 weeks of age and then kept in institutional animal house for acclimatization for 1 week. Mice were then randomized into two groups and were maintained on HFD (to mimic Obese/diabetic conditions) and control-diet for 16 weeks.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

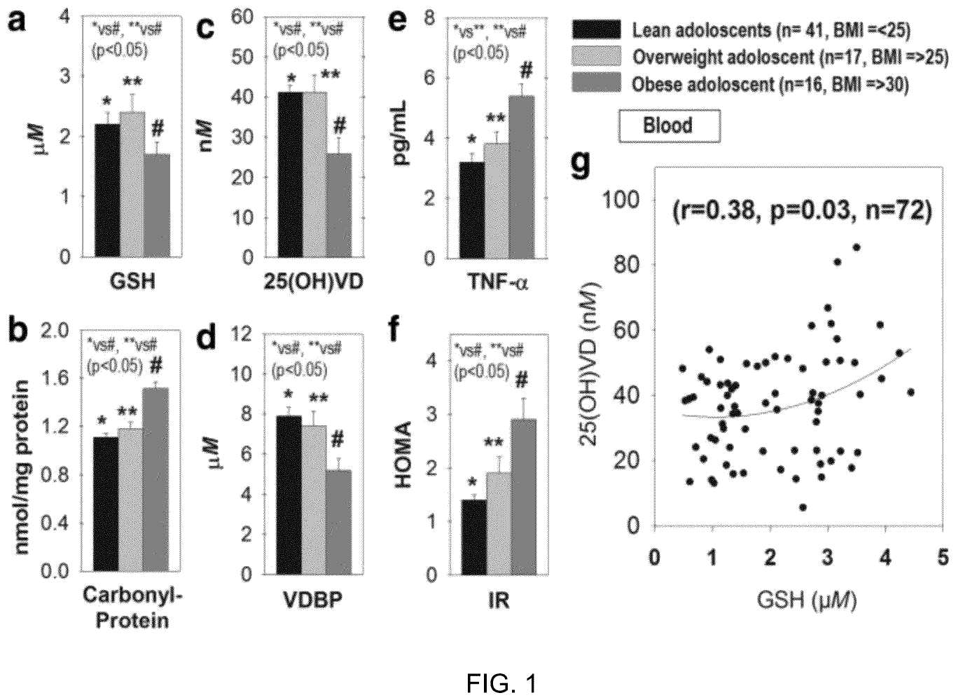

Turning now to FIGS. 1-14, a brief description concerning the various components of the present invention will now be briefly discussed. The inventors disclose experiments that demonstrate a previously undiscovered mechanism by which GSH status positively upregulates the bioavailability of 25(OH)VD, and that supplementation with a combination of VD and either LC or GSH precursor or both, rather than supplementation with VD alone, is beneficial and helps achieve more successful VD supplementation.

The inventors disclose a link between 25(OH)VD deficiency and a reduction in glutathione (GSH) in obese adolescents. The improvement in GSH status that results from co-supplementation with VD and l-cysteine (LC; a GSH precursor) significantly reduced oxidative stress in a mouse model of 25(OH)VD deficiency. It also positively upregulated VD regulatory genes (VDBP/VD-25-hydroxylase/VDR) in the liver and glucose metabolism genes (PGC-1α/VDR/GLUT-4) in muscle, boosted 25(OH)VD, and reduced inflammation and insulin resistance (IR) levels in the blood compared with supplementation with VD alone. In vitro GSH deficiency caused increased oxidative stress and downregulation of VDBP/VD-25-hydroxylase/VDR and upregulation of CYP24a1 in hepatocytes and downregulation of PGC-1α/VDR/GLUT-4 in myotubes. The inventors study demonstrates that improvement in the GSH status exerts beneficial effects on the blood levels of 25(OH)VD, as well as on the inflammation and IR in a VD-deficient mouse model. Thus, the VD supplements widely consumed by the public are unlikely to be successful unless the GSH status is also corrected.

Risk factors for 25(OH)VD deficiencies include race (darker pigmented skin tones), higher body mass index (BMI), winter season, higher geographic latitudes, and diet. Circulating 25(OH)VD is considered to be a comprehensive and stable metabolite, levels of which can be used to diagnose 25(OH)VD deficiency and monitor VD consumption. The metabolic factors responsible for the limited success of VD supplementation studies, despite the convincing association between low 25(OH)VD levels and poor health, remain unknown.

VD or cholecalciferol in the human body is derived mostly from either diet or from skin exposure to ultraviolet B from sunlight. Most people require dietary supplementation with VD to achieve the recommended blood levels of 25(OH)VD. The liver is the principal site where cholecalciferol is converted to 25(OH)VD by VD-25-hydroxylase (cytochrome p450 enzymes [CYP], CYP2R1, CYP27A1). 25(OH)VD is bound to vitamin D binding protein (VDBP) and transported into the circulation. VDBP is primarily synthesized and secreted by the liver. (CYP27B1), which converts 25(OH)VD to its active metabolite [1alpha,25-dihydroxyvitamin D3, 1,25(OH)₂VD], is present in both renal (major site) and nonrenal tissues. Even though renal tissue is considered to be a major site for 1,25(OH)VD formation, recent studies demonstrate expression of (CYP27B1) in nonrenal cells and tissues, indicating localized 1,25(OH)₂VD formation and its tissue-specific paracrine function in different tissues.

(CYP24A1) is involved in the catabolic inactivation of 1,25(OH)₂D3 and its inhibition is thought to limit 1,25(OH)₂D3 signaling. Genetic variations in VDBP/(CYP2R1) are known to influence 25(OH)VD blood levels in response to VD supplementation. Most cells have receptors for VD known as vitamin D receptor (VDR). The biological actions of 1,25(OH)₂VD are directly related to the VDR content of target tissues. Muscle is a major site of glucose metabolism and maintenance of glucose homeostasis. Therefore, biosynthesis and metabolism of VD are under the control of VD regulatory genes (GC/VDBP/VD-25-hydroxylase) in the liver, while the downstream actions of the active 1,25(OH)₂VD in muscle are mediated by glucose metabolism genes (VDR/peroxisome proliferator-activated receptor gamma coactivator 1-alpha [PGC-1α]/glucose transporter type 4 [GLUT-4]).

Glutathione (GSH) is a major antioxidant and its depletion increases oxidative stress and extensive carbonylation of proteins. Oxidative modification or carbonylation covalently modifies endogenous enzymes and proteins, which can result in the loss of protein function, insulin resistance (IR), and impaired cell function, and play a significant role in the etiology of several human diseases. Oral supplementation with GSH or l-cysteine (LC; a GSH precursor) can improve the GSH status in blood and tissues while lowering inflammation and IR in humans and animals. However, there is no report in the literature of a link between impaired GSH status and impaired status of the VD regulatory genes in the liver or glucose metabolism genes in muscle.

The inventors disclose their investigation of the dual roles of GSH in increasing circulating 25(OH)VD and augmenting the actions of active VD metabolites in one of the target tissues (skeletal muscle), which is a major site for glucose metabolism. It is further disclosed that Glutathione stimulates vitamin D regulatory genes, lowers oxidative stress and inflammation, and increases 25-hydroxy-vitamin D levels in blood. Finally, a novel approach to treat 25-hydroxyvitamin D deficiency is disclosed.

Association between GSH and 25(OH)VD blood levels in adolescents. Blood levels of GSH and 25(OH)VD were significantly lower in obese compared with lean or overweight adolescents (FIG. 1a, c). VDBP levels were significantly lower in obese children compared with both lean and overweight (FIG. 1d). Blood levels of carbonyl protein were significantly elevated in obese adolescents compared with lean and overweight, suggesting elevated oxidative stress level in obese subjects (FIG. 1b). Tumor necrosis factor alpha (TNF-α) levels and homeostatic model assessment (HOMA)-IR were significantly higher in obese compared with lean or overweight subjects (FIG. 1e, f). FIG. 1g shows a significant positive correlation between 25(OH)VD and GSH (r=0.38, p=0.03, n=72).

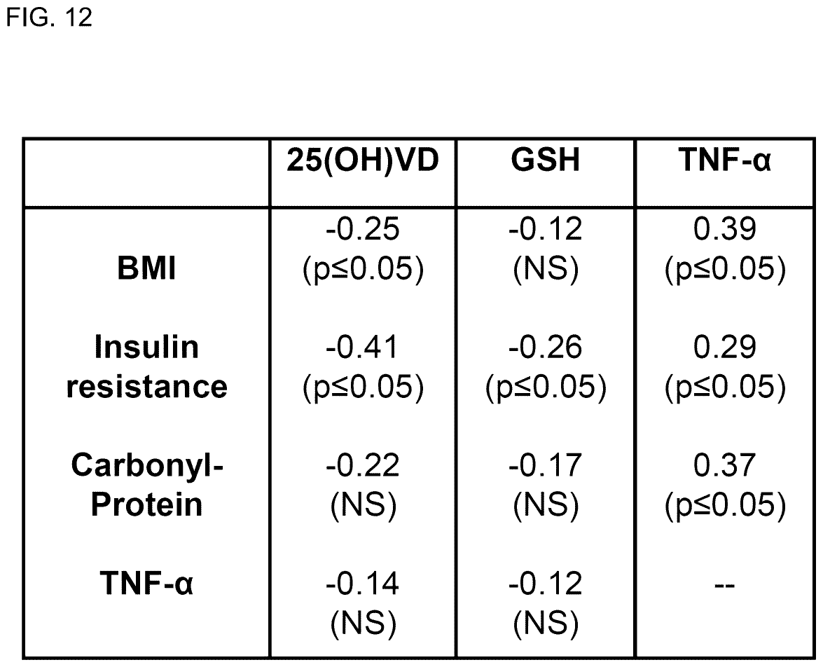

FIG. 12 shows that 25(OH)VD has a negative association with IR (r=−0.28, p=0.04); IR also showed a negative correlation with GSH (r=−0.25, p=0.05) and a positive association with TNF-α (r=0.27, p=0.04). BMI shows negative association with 25(OH)VD (r=−0.25) and positive association with TNF-α (r=0.39). TNF-α showed positive association with BMI (r=0.39), protein carbonyl (r=0.37), and IR (r=0.29). Protein carbonyl association with GSH was not significant. Ages of subjects in each group were similar, while BMI levels were significantly different in each group. Studies demonstrate low plasma levels of 25(OH)VD in humans with genetic mutation for VDBP or in VDBP-knockdown (KD) mouse models. Blood concentrations of VDBP are positively related to the half-life of circulating 25(OH)VD.

This data evidences that lower VDBP can contribute to decreased circulating 25(OH)VD levels in obese adolescents. A positive association exists between blood levels of GSH and those of 25(OH)VD. The present disclosure of a positive association between circulating 25(OH)VD and GSH status is unique and interesting because in contrast to adults, the adolescent population has a narrow age range (14-17 years) and does not have any of the confounding variables such as medications or clinical disorder. This led the inventors to search whether GSH regulates VD regulatory genes and 25(OH)VD status, and additionally whether GSH deficiency increases TNF-α levels and downregulates PGC-1α/VDR/GLUT-4 signaling of glucose metabolism.

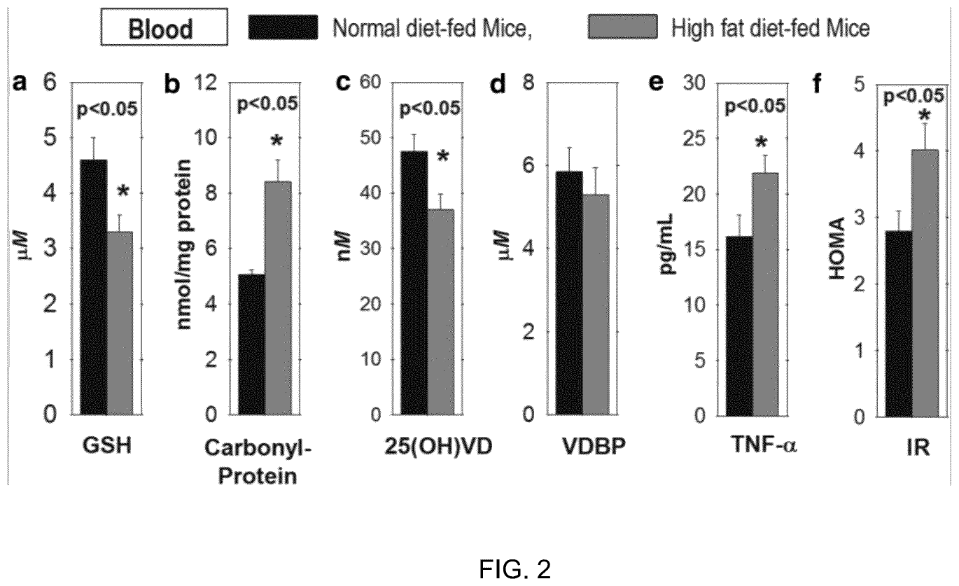

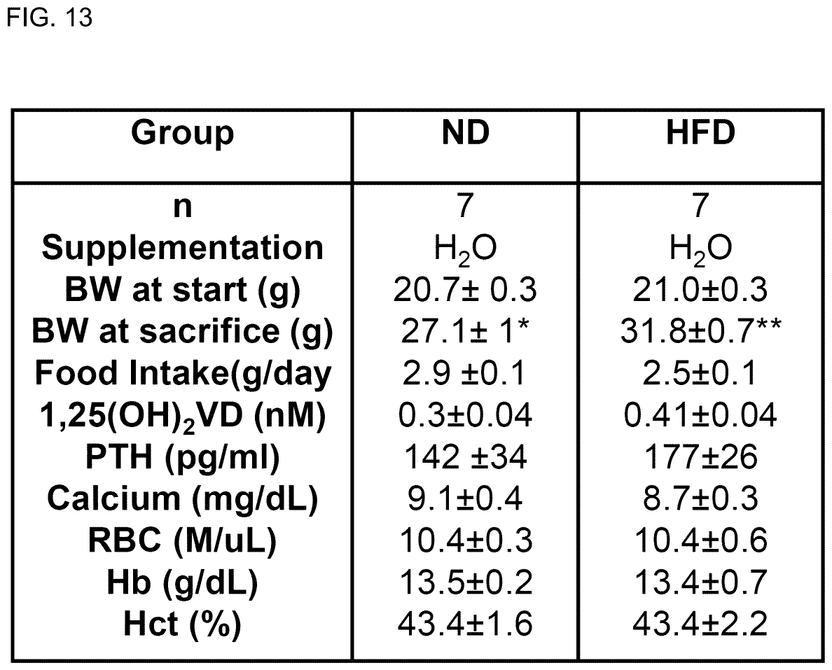

Effect of high-fat diet feeding on GSH, 25(OH)VD, and carbonyl protein levels in blood, and GSH metabolism genes and VD regulatory genes in liver and muscle in mice: Male C57BL/6J mice (5 weeks old) were purchased from The Jackson Laboratory (Bar Harbor, Me.). The animals were fed either a standard chow diet (Control: Harlan TD.08485, providing 5.2% calories as fat) or a high-fat diet (HFD) for 16 weeks. Composition of normal and HFD is given previously. Data given in FIG. 2 show that the HFD-fed mice exhibited significantly lower levels of GSH (FIGS. 2a) and 25(OH)VD (FIG. 2c) and higher levels of carbonyl protein (FIG. 2b), TNF-α (FIG. 2e), and IR (FIG. 2f) similar to the obese adolescent subjects' data. VDBP levels were not significantly different between HFD-versus control diet-fed group. Body weight (BW), food intake, parathyroid hormone (PTH), calcium, and blood count levels in the blood of mice fed normal diet and HFD mice groups were similar (FIG. 13).

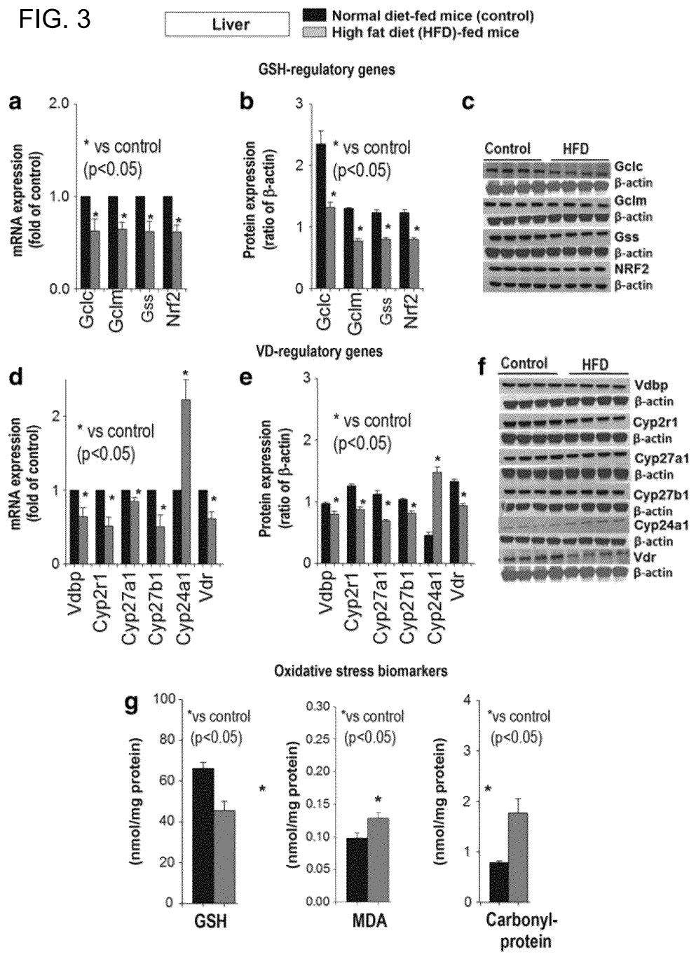

FIG. 3 shows the mRNA and protein expression of genes GCLC (glutamate-cystein ligase catalytic subunit)/GCLM (glutamate-cysteine ligase regulatory subunit)/GSS/NRF2 (nuclear factor erythroid-2-related factor) involved in GSH synthesis (FIG. 3a-c), and genes VDBP/CYP2R1/CYP27B1/VDR, which determine bioavailability of 25(OH)VD, were significantly downregulated (FIG. 3d-f) in livers of HFD-fed mice compared with normal diet-fed mice. Interestingly, mRNA and protein expression levels of CYP24A1 that degrade 25(OH)VD are upregulated in the liver of mice fed HFD in comparison with mice fed normal diet.

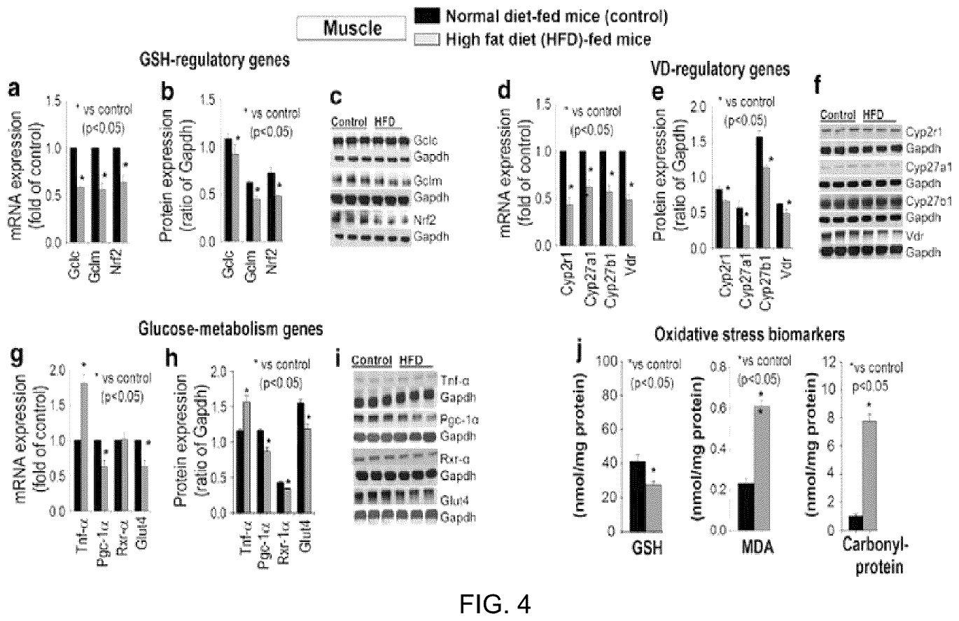

FIG. 4 shows a significant decrease in total GSH and increased oxidative stress markers (FIG. 4j) in muscle. GSH (GCLC/GCLM/NRF2) and VD regulatory genes (VDBP/CYP2R1/CYP27B1) are downregulated significantly (FIGS. 4a-f) in HFD-fed mice muscle. FIGS. 4g-i show that mRNA and protein expression of genes that regulate glucose metabolism (VDR/PGC-1α/GLUT-4) is significantly downregulated and TNF-α increased in the muscle of mice fed HFD compared with normal diet-fed mice group.

There was a significant increase in lipid peroxidation and protein oxidation with decreased GSH levels in skeletal muscle (FIG. 4j) of HFD-fed compared with normal diet-fed mice. A similar trend was observed in liver tissue (FIG. 3g). Protein-bound carbonyls are relatively more stable than lipid peroxidation products. This demonstrates that HFD consumption increases cellular oxidative stress levels.

Overall, HFD feeding resulted in a significant downregulation of genes that synthesize GSH and lower levels of GSH in blood, liver, and muscle. Similarly, there was a significant downregulation of VD regulatory genes in the liver and muscle of mice fed HFD. In addition, there was a significant downregulation of glucose metabolism genes in muscle of mice fed HFD in comparison with normal diet-fed mice group. A decrease in blood and tissue GSH reflects exhaustion or impaired antioxidant pathways and increased oxidative stress in tissues in mice consuming HFD.

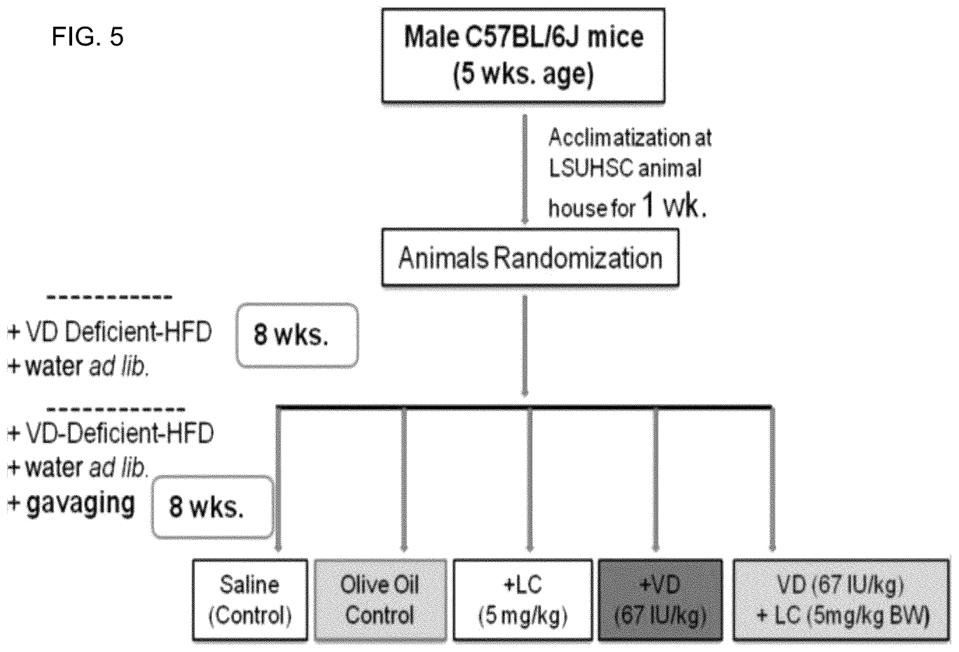

Effect of supplementation with VD along with LC on plasma levels of GSH, 25(OH)VD, and IR, and on GSH and VD regulatory genes in liver and on GSH and glucose metabolism genes in muscle: Beginning at 5 weeks of age, male C57BL/6J mice were fed and maintained on a VD-deficient HFD and water ad libitum for 16 weeks. Mice were gavaged daily for the last 8 weeks with saline, olive oil (OO), LC (5 mg/kg BW), VD (67 IU/kg BW), or LC+VD to investigate whether co-supplementation with GSH precursor has a better effect on blood levels of 25(OH)VD compared with levels achieved using VD alone. VD was dissolved in OO and one group of mice was also gavaged with OO (vehicle) alone. Details of experimental design are shown in FIG. 5.

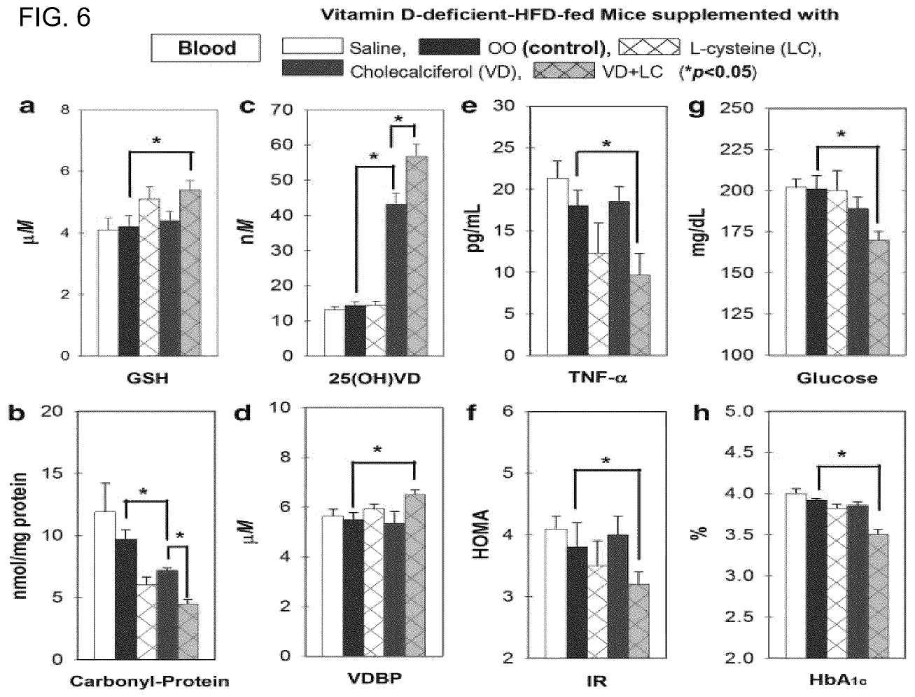

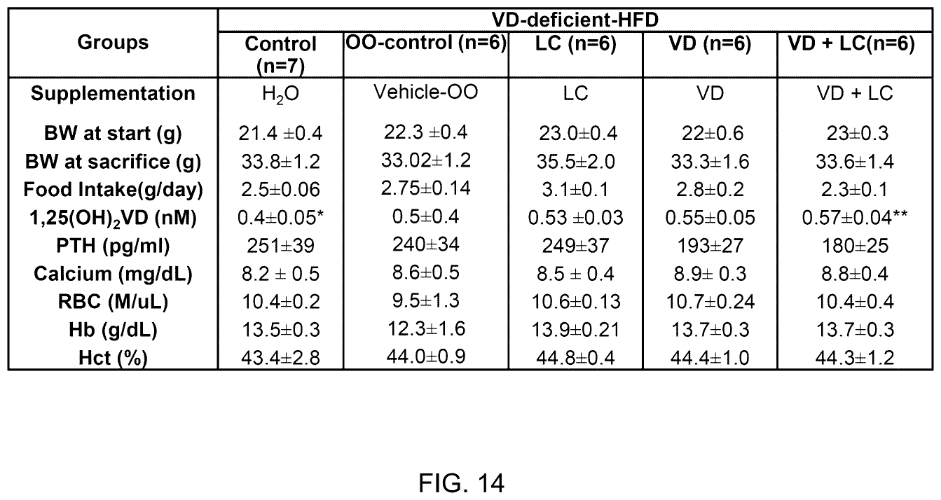

The effect of VD with and without cosupplementation with LC on blood levels of GSH, carbonyl protein, 25(OH)VD, VDBP, TNF-α, IR, glucose, and HbA_{1c} is shown in FIGS. 6a-h. VD alone did not show effect on GSH, TNF-α, VDBP, IR, and HbA_{1c}. However, supplementation with combined VD (cholecalciferol)+LC significantly corrected the GSH status and showed a significant decrease in TNF-α, IR, and HbA_{1c} in the blood compared with levels seen in the mice supplemented with OO (vehicle)-control group. In addition, supplementation with combined VD (cholecalciferol)+LC showed a greater increase in 25(OH)VD and decrease in protein carbonylation levels in the blood compared with the OO-supplemented control mice. There were no changes in food intake, RBC indices, or calcium among these groups. There was also no change in blood counts, which indicates that the decrease in HbA_{1c} values seen in the VD+LC group was not due to any effect on cell viability (FIG. 14).

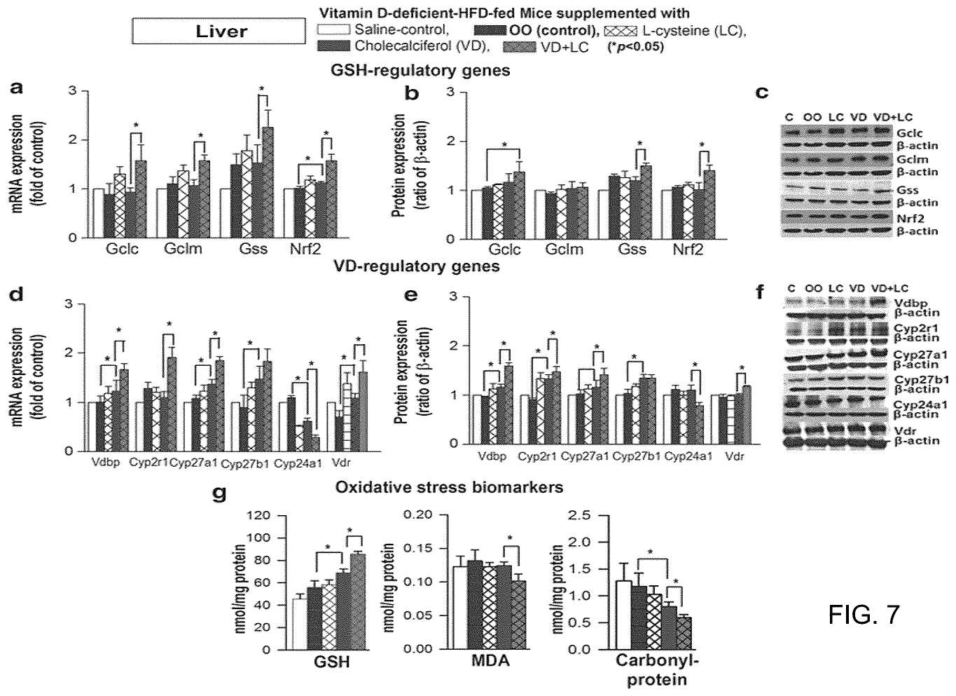

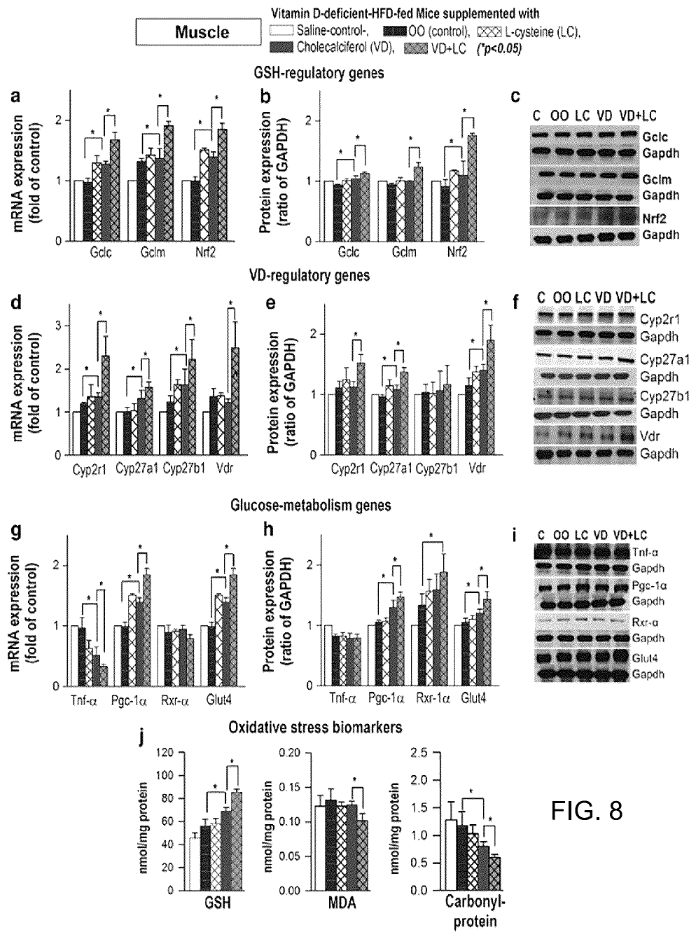

Cosupplementation with VD+LC caused a significantly greater upregulation of mRNA and protein expression of GSH synthesizing genes (GCLC/GSS/NRF2), GSH status, and VD regulatory genes (CYP2R1/CYP27A1/VDBP/VDR) in the liver (FIGS. 7a-f). In addition, CYP24A1 showed significantly lower mRNA and protein expression levels in the liver of mice supplemented with VD+LC in comparison with VD-alone mice. This study measured total 25(OH)VD status using an enzyme-linked immunosorbent assay (ELISA) kit. Studies in the literature show that 25(OH)VD analyses using either an ELISA kit or the MC/MS approach show a significant correlation. Similarly, FIG. 8 shows that there was a significant upregulation of GCLC/GCLM/NRF2 (FIG. 8a-c) and VD regulatory genes CYP2R1/CYP27A1/VDR (FIG. 8d-f) in the muscle of mice supplemented with VD+LC compared with VD alone.

In addition, there was a significant upregulation of PGC-1α/GLUT-4 (FIG. 8g-i) and GSH status (FIG. 8j) in muscle of mice supplemented with VD+LC in comparison with VD-alone supplemented mice (FIG. 8g-i). In addition, levels of protein oxidation and lipid peroxidation were significantly reduced in the liver (FIG. 7g) and muscle (FIG. 8j) of mice supplemented with VD+LC compared with those in mice supplemented with VD alone. The increase in GSH and reduction of oxidative stress biomarkers in blood and tissues reflect reduction of oxidative stress levels in tissues of mice supplemented with VD+LC compared with tissues of mice supplemented with VD alone.

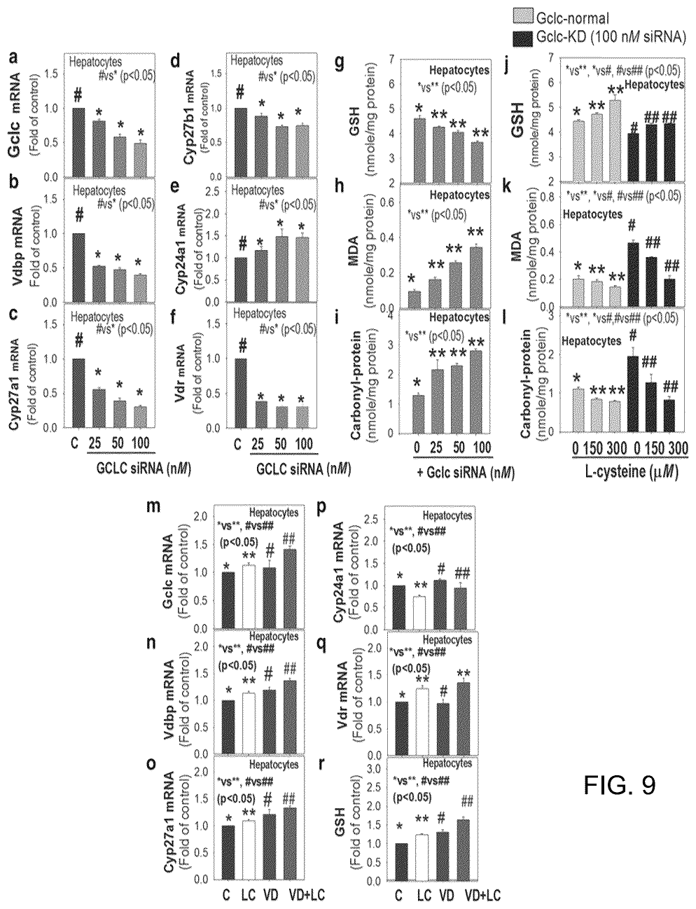

FIG. 9 shows that GCLC KD resulted in a dose-dependent decrease in GCLC (FIG. 9a) with downregulation of VDBP, CYP27A1, CYP27B1, and VDR and upregulation of CYP24A1 mRNA levels (FIGS. 9b-f) in hepatocytes. FIG. 9g shows a decrease in the level of GSH and increase in lipid peroxidation and carbonylated protein (FIGS. 9h, i) levels in GCLC-KD hepatocytes. The effect of GSH deficiency and of LC supplementation on GSH, malondialdehyde (MDA), and carbonyl protein in GCLC-normal and GCLC-KD hepatocytes is shown in FIGS. 9j-l. GSH deficiency (8-21%) resulted in significantly increased levels of carbonyl protein (77-277%) and MDA (67-212%) levels, and LC supplementation improved the status of GSH and reduced oxidation of both proteins and lipids in GSH-normal and GSH-deficient hepatocytes. This suggests that improved GSH status has the potential to prevent oxidative stress and mediate the upregulation of VD regulatory gene levels.

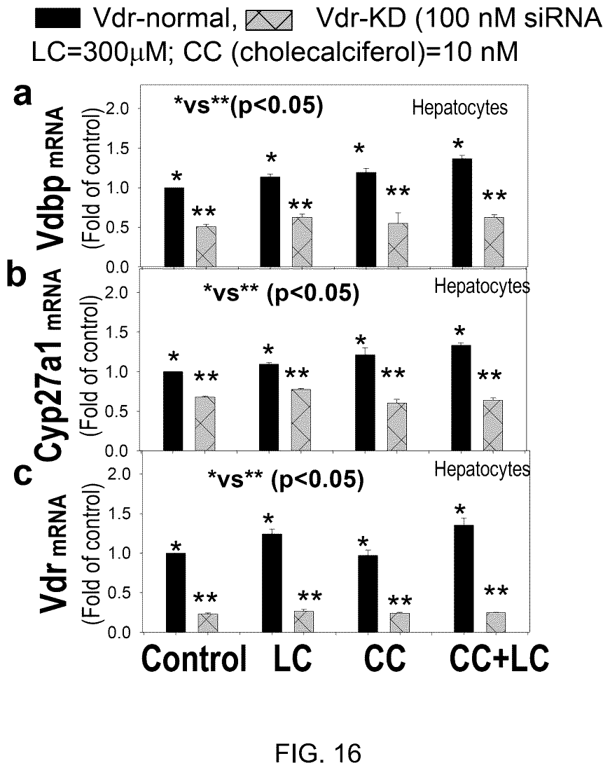

The stimulatory effect of cholecalciferol on VD regulatory genes was higher in LC co-supplemented cells, as shown in FIGS. 9m-r. GSH deficiency impairs VD regulatory gene expression, whereas co-supplementation with VD and LC can positively modify the status of GSH, CYP24A1, and VD regulatory genes in hepatocytes. These studies show that GSH status positively upregulates the gene expression of CYP27E31 that converts 25(OH)VD to 1,25(OH)₂VD. VDR-KD was induced using siRNA and was able to achieve nearly 80% VDR-KD in hepatocytes. VDR-KD caused a simultaneous decrease in VD metabolism genes CYP27A1, VDBP, and VDR, while upregulation of genes by VD+LC was abolished in VDR-KD hepatocytes (FIG. 16). Supplementation with LC alone caused upregulation of VDBP but not of the CYP27A1 gene compared with results in control VDR-KD cells (FIG. 16). This suggests that VDR mediates the beneficial effect of cholecalciferol on VD metabolism gene upregulation.

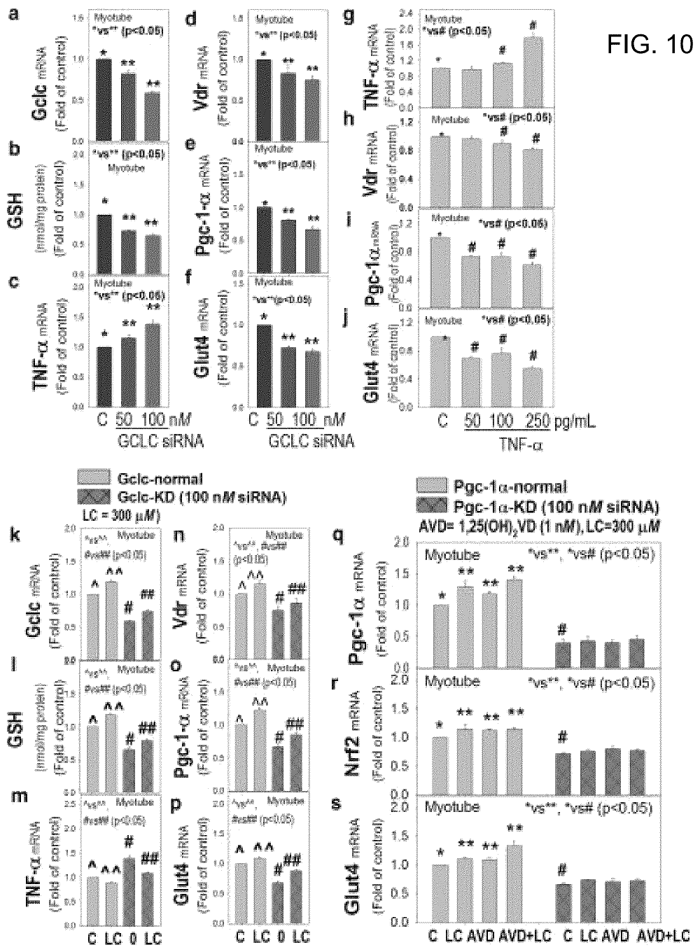

FIG. 10 shows that GSH deficiency (GCLC-KD) caused a simultaneous increase in TNF-α and decrease in VDR/PGC-1α/GLUT-4 mRNA expression in GCLC-KD C2C12 myotubes (FIGS. 10a-f). Stimulation with exogenous TNF-α per se downregulates VDR/PGC-1α/GLUT-4 in myotubes (FIGS. 10g-j). Supplementation with LC, which increases GSH levels, caused an increase in mRNA expression of VDR/GLUT-4, and a decrease in TNF-α levels in both GCLC-normal and GCLC-KD myotubes (FIGS. 10k-p). This evidences that GSH deficiency caused an increase in TNF-α and decrease in PGC-1α, as well as a decrease in the VDR/GLUT-4 needed for the action of 1,25(OH)₂VD in muscle. Deficient GSH levels can result in inflammation, which can be reversed by improving GSH status. Further studies examined the effect of combined LC and 1,25(OH)₂VD and of PGC-1α KD on GLUT-4 in myotubes.

Cotreatment with LC and 1,25(OH)₂VD (active vitamin D) resulted in significantly greater upregulation of PGC-1α/NRF2/GLUT-4 (FIG. 10q-s) gene expression in comparison with results from treatment with 1,25(OH)₂VD alone; however, this was not seen in PGC-1α-KD cells. Thus, a reduction in levels of TNF-α by LC will result in lowered inflammation and improved glucose metabolism and skeletal muscle function. 1,25(OH)₂VD was used to understand its efficacy on glucose metabolism genes in muscle cells, whereas cholecalciferol was used with hepatocyte studies to understand 25(OH)VD biosynthesis from cholecalciferol in liver.

Discussion: GSH is a major antioxidant and a cofactor of many enzymes in the human body. GSH is readily measured in blood and reflects the in vivo defense against oxidative stress. The inventors' experiments demonstrate that a reduction in GSH status is linked to 25(OH)VD deficiencies in obese adolescents and in HFD-fed mice. A decrease in blood GSH and increased oxidative stress reflect exhausted or impaired antioxidant pathways in obese humans and HFD-fed mice. Lower levels of GSH can occur because of non-availability of LC from food consumption, increased ROS production and oxidative stress from energy-rich diet consumption, and/or increased utilization of GSH relative to its biosynthesis. The depletion or deficiency of GSH can increase oxidative stress and extensive carbonylation of proteins, which can increase inflammatory mediators such as TNF-α and impair normal function of endogenous enzymes and proteins, and IR.

The inventors' experiments show a significant positive correlation between 25(OH)VD and GSH status in adolescents. This led the inventors to examine whether GSH regulates VD regulatory genes and 25(OH)VD status in the blood. Using the mouse model of 25(OH)VD deficiency, the inventors showed a significantly greater increase in blood 25(OH)VD levels after co-supplementation with VD (cholecalciferol)+LC (a GSH precursor) compared with results in a group supplemented with VD alone. In addition, supplementation with VD alone does not affect GSH, TNF-α, or IR levels in blood; however, when compared with results in the control group, co-supplementation using VD with LC significantly increases GSH levels and reduces oxidative stress, TNF-α, and IR levels in blood, and increases GSH levels and reduces oxidative stress in liver and muscle. Transfection studies demonstrate that GSH deficiency causes increased oxidative stress, downregulation of VDBP/VD-25-hydroxylase/VDR, and upregulation of CYP24A1 in mouse hepatocytes and downregulation of PGC-1α, VDR, GLUT4 in mouse myotubes, similar to results seen in HFD-fed mice.

Improvement in GSH status by LC prevented the downregulation of VD regulatory genes in hepatocytes and glucose metabolism genes in myotubes. These in vitro and in vivo experiments demonstrate the dual roles of GSH in increasing circulating 25(OH)VD and augmenting the actions of active VD metabolites in one of the target tissues (skeletal muscle), which is a major site for glucose metabolism.

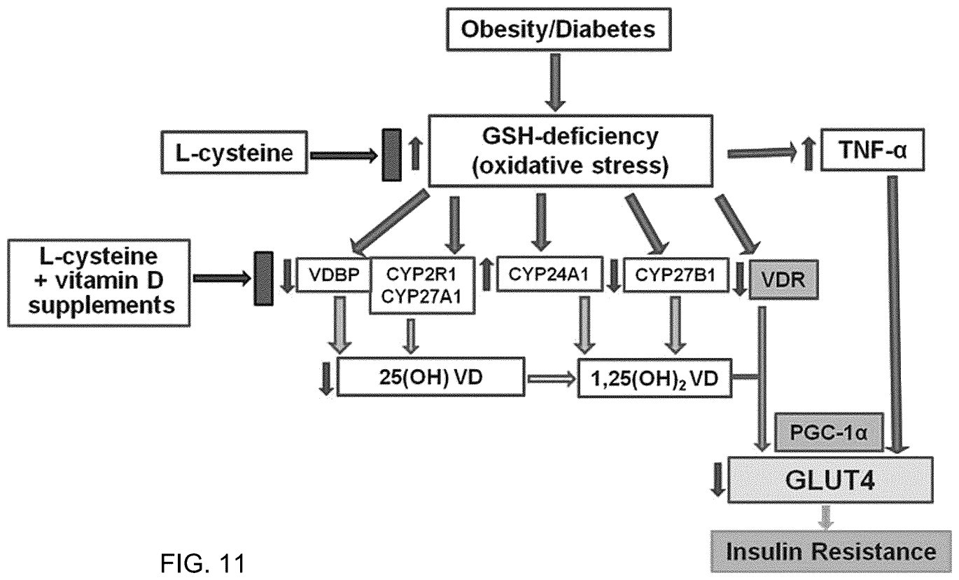

FIG. 11 outlines the proposed mechanism for role of GSH deficiency in 25(OH)VD deficiency and potential of combined VD and LC supplementation on stimulation of VD regulatory genes and protection from 25(OH)VD deficiency and inflammation. The mechanism potentially responsible for the increased blood levels of 25(OH)VD could be that LC upregulates the synthesis of GSH, thus improving the status of GSH, which reduces oxidative stress and prevents impaired (reduced) levels of VDBP/VD-25-hydroxylase/VDR, thereby helping protect the status of 25(OH)VD levels. VDBP is required for efficient transport and VD-25-hydroxylase is needed for the hydroxylation of cholecalciferol.

Furthermore, upregulation of VDR status in target tissues stimulates the translocation of the VDR/1,25(OH)₂VD complex to the nucleus for activation of the VDR/PGC-1α/GLUT-4 pathway responsible for metabolic actions of 1,25(OH)₂VD. The PGC-1α is an inducible transcriptional coactivator that upregulates expression of GLUT-4 in skeletal muscle and is a coactivator of the retinoic X receptor (RXRα). PGC-1α functions as a cofactor for NRF2, which is implicated in the biosynthesis of GSH. Therefore, upregulation of PGC-1α and GLUT-4 is beneficial in reducing IR and glycemia.

This study provides evidence for a previously undiscovered mechanism that describes how 25(OH)VD deficiency/inadequacy is linked to lower GSH levels and that boosting GSH status beneficially upregulates the genes of VD metabolism and VDR, both of which are needed to increase the bioavailability and blood levels of 25(OH)VD and reduce inflammation levels. GSH provides not only upregulation of VD regulatory genes but also adds to GLUT-4 activation. Thus, increasing GSH status by combined supplementation with LC or a GSH precursor along with VD provides a novel approach to treat widespread 25(OH)VD deficiency/inadequacy in populations worldwide.

Investigation of mRNA expression and protein expression analyses of genes showed some differences between mRNA expression and protein expression levels. This could be due to variances in post-transcription regulation; differences in mRNA and protein turnover rates across the spectrum of genes involved; or differences in the technical precision of the methodology used. The inventors demonstrate herein that the improvement in the GSH status exerts measurable and beneficial effects on both mRNA and protein expression levels of VDBP/VD-25-hydroxylase/VDR as well as PGC-1α/GLUT-4 genes. Blood levels of 25(OH)VD are lower in humans with a genetic mutation for VDBP or in VDBP-KD mouse models. Blood concentrations of VDBP are positively related to the half-life of circulating 25(OH)VD. This evidences that lower circulating 25(OH)VD levels could be a result of the decreased VDBP levels seen in obese adolescents. GSH is formed from LC by the enzymatic action of glutamate-cysteine ligase (GCL) and GSH synthetase. lc metabolism is facilitated by the LC transporter. Whether HFD feeding has any effect on the status of the LC transporter in tissues is not known.

Vitamin D helps the body absorb calcium and maintain bone and muscle health. Evidence in the literature supports the positive link between a high consumption of milk products and leafy vegetables and biomarkers of bone health and 25(OH)VD levels in the blood. In fact, milk and leafy vegetables are rich sources of both vitamin D and of GSH and methionine/lc. This may explain why consumption of food rich in lc/methionine and GSH can increase the bioavailability of VD and improve the quality of life. In addition, a large prospective E3N-Epic cohort study conducted among 64,233 middle-aged women reported that women consuming a diet with higher levels of total antioxidant capacity were found to have a lower risk for type 2 diabetes. The inventors' experiments suggest that there is an interaction between the consumption or status of the dietary nutrients LC/methionine and an enhanced bioavailability of vitamin D (cholecalciferol). The use of lower VD doses combined with a either GSH or one or more GSH precursor(s), or both, provides a novel approach to correct 25(OH)VD deficiency/inadequacy. This research provides evidence for the recommendation to use a combination of VD and a GSH precursor for supplementation, rather than VD alone, to achieve greater success with the VD supplements widely used by the public in pursuit of better health.

These findings focus attention on the fact that the VD supplements widely consumed by the public are unlikely to be efficacious unless the status of the VD metabolism genes is improved by first correcting the status of GSH. This suggests that combined consumption of GSH precursors and VD, rather than solely using high-dose VD, is both novel and a better strategy with which to achieve a more efficacious bioavailability in response to cholecalciferol consumption and to increase blood levels of 25(OH)VD.

Materials and Methods. Enrollment of human subjects: The inventors' experiments were carried out after informed written consent was obtained from all subjects according to the protocol approved by the Louisiana State University Health Sciences Center (LSUHSC) Institutional Review Board (IRB). This study enrolled adolescent boys and girls ages 14-17 years, in good health (other than being overweight or IR), who provided written informed assent and parental consent. Inclusion criteria were children who are not smoking or taking any medicines, food supplements, or antihistamines. All subjects who gave written informed consent were invited to return to have blood drawn after fasting overnight (8 h). The exclusion criterion was that subjects were excluded if pregnant or suspected to be pregnant, applied only to female subjects. Urine was collected from the female subjects for urine pregnancy testing. Subjects were allowed to sit quietly for 10 min after which blood was collected from an easily accessible forearm vein using a butterfly needle and Vacutainer® tube. Ethylenediaminetetraacetic acid (EDTA) blood tubes were brought to the research laboratory. Isolated plasma was stored in different aliquots and frozen immediately at −80° C. for analyses of the biochemical parameters.

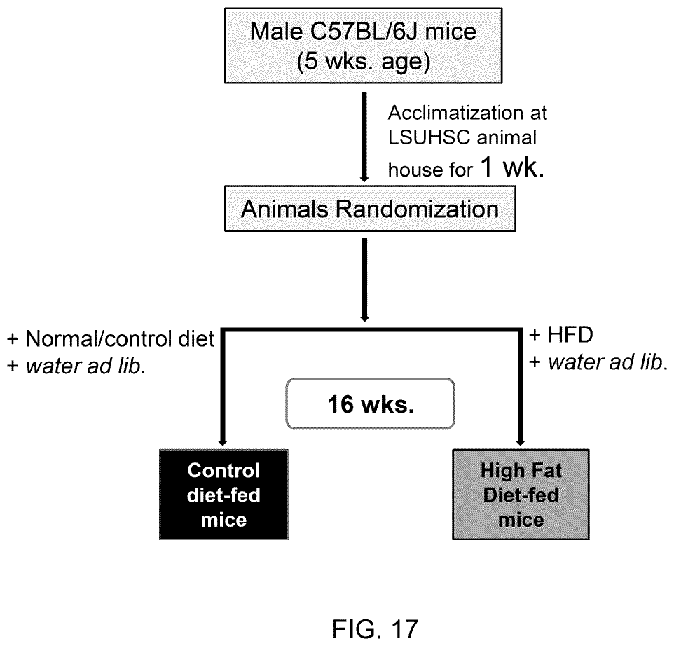

Animal studies. Male C57BL/6J mice (5 weeks old, 20-24 g) were purchased from The Jackson Laboratory and acclimatized in the institutional animal house for 1 week. Mice were divided into various groups by computer-generated randomization and then housed and labeled in individual cages. They were fasted overnight and then weighed. Blood glucose was assessed by tail prick using an Accu-Chek glucometer (Boehringer Mannheim Corp., Indianapolis, Ind.). BW and blood glucose were monitored weekly. Control animals were fed a normal diet (lower in fat), while animals in the HFD group were fed HFD for 16 weeks. Flow diagram showing details of diet feeding of mice is given in FIG. 17. For studies with VD-deficient animals, the mice were maintained on a VD-deficient HFD for 16 weeks. After 8 weeks, the mice were supplemented by oral gavage for another 8 weeks with either 5 mg LC/kg BW daily (LC) or 67 IU VD/kg BW (+VD), or the same doses of cholecalciferol and l-cysteine (LC+VD) (FIG. 5).

In addition, two groups of mice maintained on the VD-deficient HFD were also simultaneously supplemented by oral gavage with either water or the same dose of the vehicle used for dissolving cholecalciferol (OO) (FIG. 5). The animals were maintained under standard housing conditions at 22° C. 2° C. with 12/12-h light/dark cycles. Normal diet, HFD, and VD-deficient HFD were purchased from The Jackson Laboratory. The amount of food intake was monitored at 12 and 16 weeks into the treatment period to assess consumption. At the end of 16 weeks, the animals were fasted overnight and then euthanized for analysis by exposure to isoflurane (Webster Veterinary Supply, Inc., Devens, Mass.). Blood was collected via heart puncture with a 19^{1/2}-gauge needle into heparinized Vacutainer tubes. Plasma was isolated after centrifuging the blood in a 4° C. centrifuge at 3000 rpm for 10 min. The livers were perfused with cold saline to free them of residual blood. Liver and gastrocnemius muscle were collected immediately, weighed, quickly diced, and frozen in liquid nitrogen at −80° C.

Dose justification for LC and vitamin D: While lc can be taken as a supplement, it is also formed in the body from methionine. An adult ingests about 500 mg LC from dietary sources, assuming that an average protein intake is 90 g/day and that LC is about 0.6% of total protein. Similarly, taking into account daily dietary intake and diet composition, mice consume 3-5 mg LC/kg BW. The LC/methionine content of protein varies with the source of the protein. The LC dose used in our studies, 5 mg/kg BW, is theoretically a supplementation onefold to twofold that of the LC ordinarily consumed by the mice, which could be considered both modest and safe. The VD dose used was 67 IU/kg/day (1.67 μg/kg/day). Cholecalciferol was dissolved in 0.1% 00 and a stock solution of 1.67 μg/mL was prepared. An aliquot of 0.1 mL of the stock solution was given per 100 g BW using oral gavage on alternate days for 8 weeks. For alternate day gavaging, the supplementation dose was doubled to maintain a similar dose per day. The vehicle-OO control group is included in the treatment groups.

Cell culture and treatment: FL83B mouse hepatocytes (ATCC®, Manassas, Va.) were cultured and maintained in F-12K complete medium. Mouse C2C12 myoblasts were cultured at 37° C. in an atmosphere of 5% CO₂ in growth medium (GM) consisting of Dulbecco's modified Eagle's medium (DMEM) supplemented with heat-inactivated 10% fetal bovine serum and antibiotics (penicillin and streptomycin). Differentiation of myoblasts into myotubes was induced when the cells had achieved 90-95% confluence by switching the medium from GM to differentiation medium consisting of DMEM supplemented with 2% horse serum (5 days), then treated as described in the figures. siRNAs were purchased from Santa Cruz Biotechnology, Inc. (Dallas, Tex.), catalog numbers sc-41979 (GCLC), sc-36811 (VDR), and sc-38885 (PGC-1α). The control siRNA, a scrambled nonspecific RNA duplex that shares no sequence homology with any of the genes, was used as a negative control.

Cells were transiently transfected with 0-100 nM siRNA complex using Lipofectamine™2000 transfection reagent (Invitrogen, Carlsbad, Calif.). The next day cells were treated with LC (0-300 μM) for 6 h for LC-alone experiments. Pretreatment of the cells, maintained at a concentration of 1×10⁶/mL media, was done for 2 h with LC (0-300 μM), followed by treatment for 22 h with cholecalciferol or 25(OH)VD (10 nM). TNF-α (0-250 μg/mL) was exposed for 6 h to differentiated myotubes.

Justification for use of FL83B mouse hepatocytes and mouse C2C12 myoblasts: Liver is a major player in the synthesis and secretion of VDBP and the hydroxylation of cholecalciferol (vitamin D3) to 25-hydroxy-vitamin D. FL83B mouse hepatocytes express VDBP, CYP27A1, CYP27B1, CYP24A1, and VDR, but expression of CYP2R1 is very low and could not be accurately quantitated. However, the inventors obtained reproducible gene analysis results for expression of VDBP, CYP27A1, CYP27B1, CYP24A1, and VDR genes using FL83B mouse hepatocytes. Liver contains both CYP27A1 and CYP2R1 and participates in the conversion of cholecalciferol (vitamin D3) to 25-hydroxy-vitamin D. The population studies have also shown a link between CYP2R1, GC (VDBP), CYP24A1, and VDR with that of circulating 25(OH)VD concentrations. Recent studies have shown a regulatory role of CYP27A1 gene expression on the blood concentrations of 25(OH)VD.

Thus, mouse hepatocytes used in the disclosed experiments have much strength and can be used for investigating the link between GSH-deficiency, oxidative stress, and VD regulatory genes. Muscle is a major site for glucose metabolism. Differentiation of myoblasts into myotubes is very reproducible and these cells express the glucose metabolism genes such as PGC-1α, RXRα, VDR, and GLUT-4. GLUT-4 is a master regulator for the maintenance of glucose metabolism. Thus, mouse C2C12 myoblasts have much strength to investigate the role of GSH deficiency in regulation of glucose metabolism pathways.

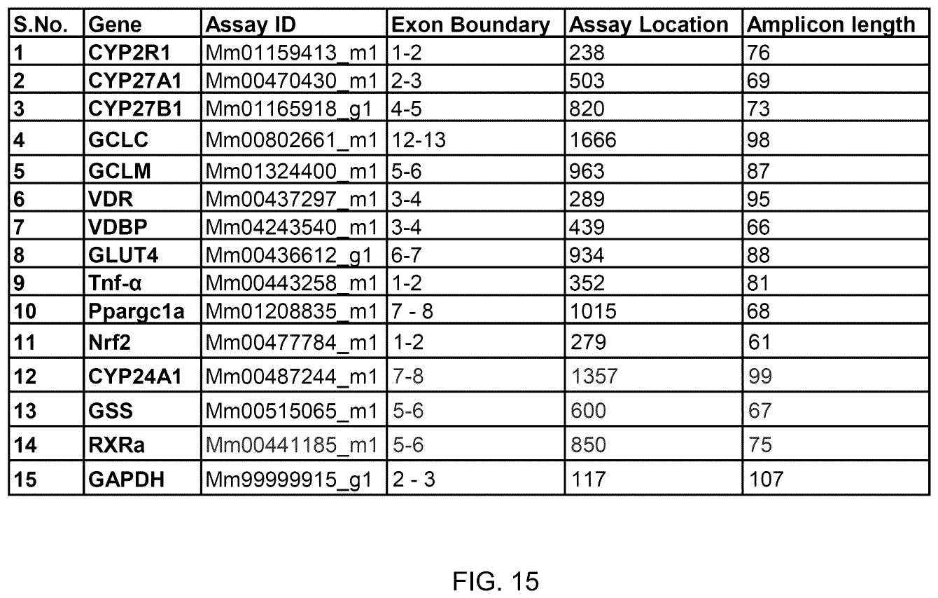

Analysis of mRNA expression using quantitative polymerase chain reaction: Total RNA was extracted from cells or tissue using the TRIzol reagent (Life Technologies) following the manufacturer's instructions. The quality and quantity of the extracted RNA were determined on a NanoDrop spectrophotometer (Thermo Scientific). First-strand complementary DNA (cDNA) synthesis was performed using a commercially available High Capacity RNA-To-cDNA kit (Life Technologies) in a final reaction volume of 20 μL. Amplification of cDNA was performed on a 7900HT Real Time polymerase chain reaction (PCR) system (Applied Biosystems). PCR conditions were 2 min at 50° C., 10 min at 95° C., 40 cycles of 95° C. for 15 s, and then 60° C. for 60 s. Details of the TaqMan-FAM-labeled primer/probe used are given in FIG. 15. Glyceraldehyde-3-phosphate dehydrogenase (GAPDH) was used as a housekeeping gene to normalize threshold cycle (CT) values.

To exclude nonspecific amplification and/or the formation of primer dimers, control reactions were performed in the absence of target cDNA. All of the experiments were run in triplicate. The relative amounts of mRNAs were calculated using the relative quantification (ΔΔCT) method. FIG. 15 gives details of primer used in the inventors' experiments.

Western blot analysis: The tissue homogenates were processed for immunoblotting studies. To extract protein from liver and gastrocnemius muscle, ˜100 mg of tissue was homogenized in RIPA buffer on ice using a rotor/stator. RIPA buffer (50 mM Tris pH 8, 150 mM NaCl, 1% NP-40, 0.5% deoxycholic acid, and 0.1% SDS) was supplemented with protease and phosphatase inhibitors (1 mM phenylmethylsulfonyl fluoride, 5 μg/mL leupeptin, 2 μg/mL aprotinin, 1 mM EDTA, 10 mM NaF, and 1 mM NaVO4). Lysates were then centrifuged for 10 min at 10,000 g at 4° C. Supernatants were collected and the protein concentrations were determined using a BCA assay kit (Pierce/Thermo Scientific, Rockford, Ill.) for Western blot analysis and high performance liquid chromatography (HPLC) assay. Equal amounts (20 μg) of proteins were separated on 10% sodium dodecyl sulfate—polyacrylamide gel electrophoresis (SDS-PAGE) and transferred to a polyvinyl difluoride membrane. Membranes were blocked at room temperature for 2 h in a blocking buffer containing 1% bovine serum albumin to prevent nonspecific binding and then incubated with an appropriate primary antibody at 4° C. overnight.

The membranes were washed in TBS-T (50 mMTris-HCl, pH 7.6, 150 mM NaCl, 0.1% Tween 20) for 30 min and incubated with an appropriate Horseradish peroxidase-conjugated secondary antibody (1:5000 dilution) for 2 h at room temperature. The protein bands were detected using ECL detection reagents (Thermo Scientific) and exposed on blue X-ray film (Phenix Research Products, Candler, N.C.). The technical replicates (n=2) and biological replicates (n=4) were done in all our immunoblot experiments. Western blot scans were analyzed using ImageJ software (developed by Wayne Rasband, National Institutes of Health, Bethesda, Md. Densitometry analyses of Western blots were normalized with respect to β-actin or GAPDH (ratio).

25(OH)VD, 1,25(OH)₂VD, VDBP, GSH, TNF-α, PTH, insulin, glucose, protein carbonyl, and MDA assays: Plasma levels of 25(OH) vitamin D were determined using an ELISA kit (Calbiotech, Spring Valley, Calif.) and 1,25(OH)₂ vitamin D using another ELISA kit (My BioSource, San Diego, Calif.). Plasma VDBP quantification was carried out using a kit purchased from ALPCO Diagnostics (Salem, N.H.). TNF-α was measured using an ELISA kit from R&D Systems (Minneapolis, Minn.). PTH (1-84) and insulin were determined using ELISA kits from ALPCO Diagnostics, and the HOMA-IR index was calculated. VDBP was measured using polyclonal antibodies (DRG Instruments, Springfield, N.J.). The kit included polyclonal antibodies that detect total VDBP levels. In the ELISA, control samples were analyzed each time to check the variation from plate to plate on different days of analysis. Protocols as given in the manufacturer's instructions were followed using appropriate controls and standards.

Levels of GSH in plasma, tissues, and cultured cells were determined using HPLC. This assay determines total GSH status. Cell viability was determined using the Alamar Blue method (Alamar Biosciences, Sacramento, Calif.). Oxidative stress was assessed by the quantification of protein carbonyls and MDA using Protein Carbonyl Colorimetric and TBARS Assay Kits, respectively (Cayman Chemical, Ann Arbor, Mich.). Measurements of HbA_{1c}, Complete Blood Count, glucose, and calcium were done at the clinical chemistry laboratories of LSUHSC-Shreveport. Due to limited amount of blood collected from each mouse, we borrowed diluents from the clinical laboratory and diluted the blood before taking it to the clinical laboratory, which reduced the amount of blood required for clinical tests. All chemicals were purchased from Sigma Chemical Co. (St. Louis, Mo.) unless otherwise mentioned.

Pharmaceutical Compositions: The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8th Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration: The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings: The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration: Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery: Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

Dosing Regimes: The present methods for treating VD deficiency associated conditions are carried out by administering a therapeutic for a time and in an amount sufficient to result in decreased insulin resistance (IR), inflammation, blood TNF-α level, blood glucose level, blood HbA1c level, and/or increased 1alpha,25-dihydroxyvitamin D3 (1,25(OH)₂VD) blood level.

The amount and frequency of administration of the compositions can vary depending on, for example, what is being administered, the state of the patient, and the manner of administration. In therapeutic applications, compositions can be administered to a patient suffering from a VD deficiency associated condition in an amount sufficient to relieve or least partially relieve the symptoms of the VD deficiency associated condition and its complications. The dosage is likely to depend on such variables as the type and extent of progression of the VD deficiency associated condition, the severity of the VD deficiency associated condition, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the VD deficiency associated condition or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

Kits: Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the VD deficiency associated condition.

Statistical analysis: Data from clinical, cell culture, and mouse studies were analyzed using regression analyses and ANOVA with Sigma Stat software (SPSS, Chicago, Ill.). A p-value of ≤0.05 for a statistical test was considered significant.

### Abbreviations Used

1,25(OH)₂VD—1alpha,25-dihydroxyvitamin D3

25(OH)VD—25-hydroxyvitamin D

ANOVA—analysis of variance

BMI—body mass index

BW—body weight

cDNA—complementary DNA

CT—threshold cycle

CYP—cytochrome P450 enzymes

DMEM—Dulbecco's modified Eagle's medium

EDTA—ethylenediaminetetraacetic acid

ELISA—enzyme-linked immunosorbent assay

GAPDH—glyceraldehyde-3-phosphate dehydrogenase

GCL—glutamate-cysteine ligase

GCLC—glutamate-cysteine ligase catalytic subunit

GCLM—glutamate-cysteine ligase regulatory subunit

GLUT-4—glucose transporter type 4

GSH—glutathione

GM—growth medium

HFD—high-fat diet

HOMA—homeostatic model assessment

HPLC—high performance liquid chromatography

IR—insulin resistance

KD—knockdown

LC—l-cysteine

MDA—malondialdehyde

NRF2—nuclear factor erythroid-2-related factor

OO—olive oil

PCR—polymerase chain reaction

PGC-1α—peroxisome proliferator-activated receptor gamma coactivator 1-alpha

PTH—parathyroid hormone

RXRα—retinoic X receptor

TNF-α—tumor necrosis factor alpha

VD—vitamin D

VDBP—vitamin D binding protein

VDR—vitamin D receptor

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of treating a cholecalciferol (VD) deficiency associated condition in a patient comprising:
administering a pharmacologically effective does of a pharmaceutical composition containing VD and one of glutathione (GSH) and a GSH precursor.

2. The method of claim 1 where the VD deficiency associated condition is one of insulin resistance (IR), inflammation, decreased 1alpha,25-dihydroxyvitamin D3 (1,25(OH)₂VD) blood level, elevated blood TNF-α level, elevated blood glucose level, elevated blood HbA_{1c} level, and one or more chronic metabolic diseases.

3. The method of claim 2 wherein the chronic metabolic disease includes one or more of obesity, diabetes, cardiovascular disease, and liver disease.

4. The method of claim 1 wherein the pharmaceutical composition contains a GSH precursor.

5. The method of claim 4 wherein the GSH precursor is one of N-acetylcysteine, l-cysteine (LC), cystathionine, homocysteine, S-adenosylmethionine, and l-methionine.

6. The method of claim 1 wherein the pharmaceutical composition is administered via one of topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, nebulized, by suppositories, and oral administration.

7. The method of claim 1 wherein the dosage of the one of GSH and GSH precursor is between 1.0 mg/kg and 20 mg/kg body weight of patient.

8. The method of claim 1 wherein the dosage of the one of GSH and GSH precursor is between 2.5 mg/kg and 10 mg/kg body weight of patient.

9. The method of claim 1 wherein the dosage of the one of GSH and GSH precursor is between 4.0 mg/kg and 6.0 mg/kg body weight of patient.

10. The method of claim 1 wherein the dosage of VC is between 0.40 μg/kg and 7.00 μg/kg body weight of patient.

11. The method of claim 1 wherein the dosage of VC is between 0.80 μg/kg and 3.50 μg/kg body weight of patient.

12. The method of claim 1 wherein the dosage of VC is between 1.60 μg/kg and 1.74 μg/kg body weight of patient.

13. The method of claim 1 wherein the dosage of the one of GSH and GSH precursor is one of between 200 mg and 2000 mg, between 350 and 1000 mg, between 450 mg and 550 mg, and 500 mg.

14. A pharmaceutical composition for treating a cholecalciferol (VD) deficiency associated condition comprising:
a pharmacologically effective does of VD and one of glutathione (GSH) and a GSH precursor.

15. The pharmaceutical composition of claim 14 wherein the pharmaceutical composition contains a GSH precursor.

16. The pharmaceutical composition of claim 15 wherein the GSH precursor is one of N-acetylcysteine, l-cysteine (LC), cystathionine, homocysteine, S-adenosylmethionine, and l-methionine.

17. The pharmaceutical composition of claim 14 wherein the pharmaceutical composition formulated for administration via one of topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, nebulized, by suppositories, and oral administration.

18. The pharmaceutical composition of claim 14 wherein dosage of the one of GSH and GSH precursor is between 200 mg and 2000 mg.

19. The pharmaceutical composition of claim 14 wherein dosage of the one of GSH and GSH precursor is between 350 and 1000 mg.

20. The pharmaceutical composition of claim 14 wherein dosage of the one of GSH and GSH precursor is between 450 mg and 550 mg.
